(19) United States
(12) Patent Application Publication
(10) Pub. No.: US 2011/0300610 A1
(43) Pub. Date: Dec. 8, 2011

(54) Thermally Conductive Microplate

(75) Inventors: Gary Lim, San Francisco, CA (US); Quentin F. Polosky, Santa Clara, CA (US)

(73) Assignee: LIFE TECHNOLOGIES CORPORATION, Carlsbad, CA (US)

(21) Appl. No.: 13/087,134

(22) Filed: Apr. 14, 2011

Related U.S. Application Data
(63) Continuation of application No. 11/623,695, filed on Jan. 16, 2007, now abandoned.
(60) Provisional application No. 60/760,895, filed on Jan. 20, 2006.

(51) Int. Cl. C12M 1/00

(52) U.S. Cl. 435/283.1

(57) ABSTRACT

Embodiments according to the present teachings of a microplate comprising a main body portion having a first surface and an opposing second surface are disclosed. A plurality of wells are formed in the first surface, each of the plurality of wells being sized to receive an assay therein. A backing is coupled to the opposing second surface of the main body portion.

### CROSS REFERENCE TO RELATED APPLICATION

This application claims a priority benefit under 35 U.S.C. §119(e) from U.S. Provisional Application No. 60/760,895, filed Jan. 20, 2006, and U.S. patent application Ser. No. 11/623,695, filed Jan. 16, 2007, which are incorporated herein by reference.

### INTRODUCTION

Currently, genomic analysis, including that of the estimated 30,000 human genes is a major focus of basic and applied biochemical and pharmaceutical research. Such analysis may aid in developing diagnostics, medicines, and therapies for a wide variety of disorders. However, the complexity of the human genome and the interrelated functions of genes often make this task difficult. There is a continuing need for methods and apparatus to aid in such analysis.

### DRAWINGS

The skilled artisan will understand that the drawings, described herein, are for illustration purposes only. The drawings are not intended to limit the scope of the present teachings in any way.

FIG. 1 is a top perspective view illustrating a microplate in accordance with some embodiments;

FIG. 2 is a top perspective view illustrating a microplate in accordance with some embodiments;

FIG. 3 is an enlarged perspective view illustrating a microplate in accordance with some embodiments comprising a plurality of wells comprising a circular rim portion;

FIG. 4 is an enlarged perspective view illustrating a microplate in accordance with some embodiments comprising a plurality of wells comprising a square-shaped rim portion;

FIG. 5 is a top perspective view illustrating a microplate having a backing in accordance with some embodiments;

FIG. 6 is a bottom perspective view illustrating the microplate;

FIG. 7 is a top perspective view illustrating a microplate having a backing and support webs in accordance with some embodiments;

FIG. 8 is a bottom perspective view illustrating the microplate;

FIG. 9 is a cross-sectional view illustrating a microplate having through-holes and a backing according to some embodiments;

FIG. 10 is a cross-sectional view illustrating a microplate having through-holes and a laminated backing according to some embodiments; and

FIG. 11 is a cross-sectional view illustrating a microplate having wells and a backing according to some embodiments.

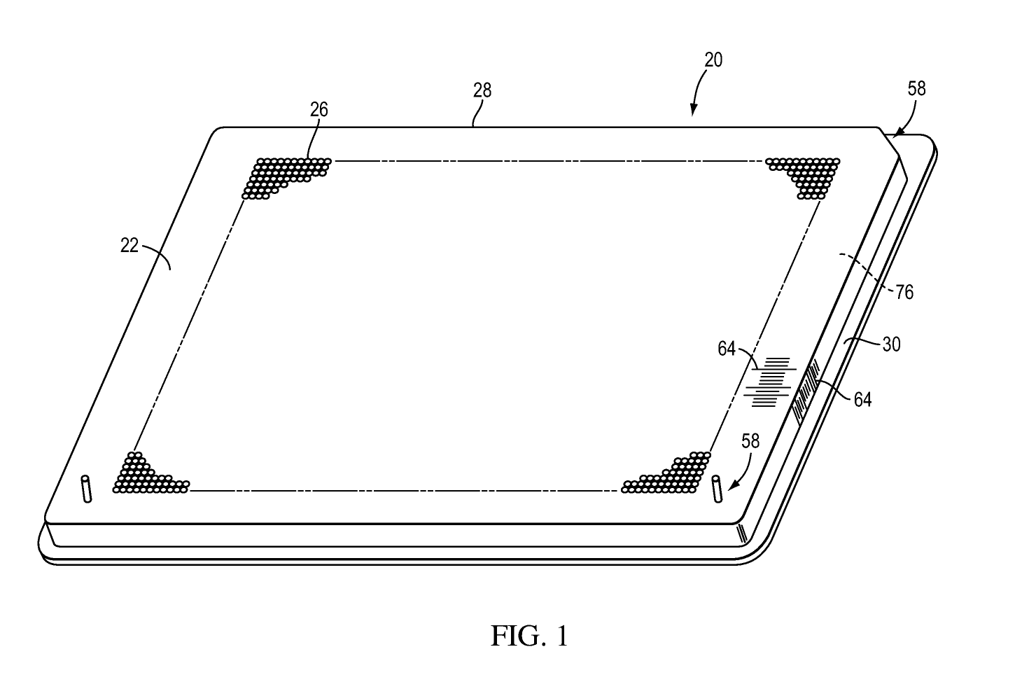

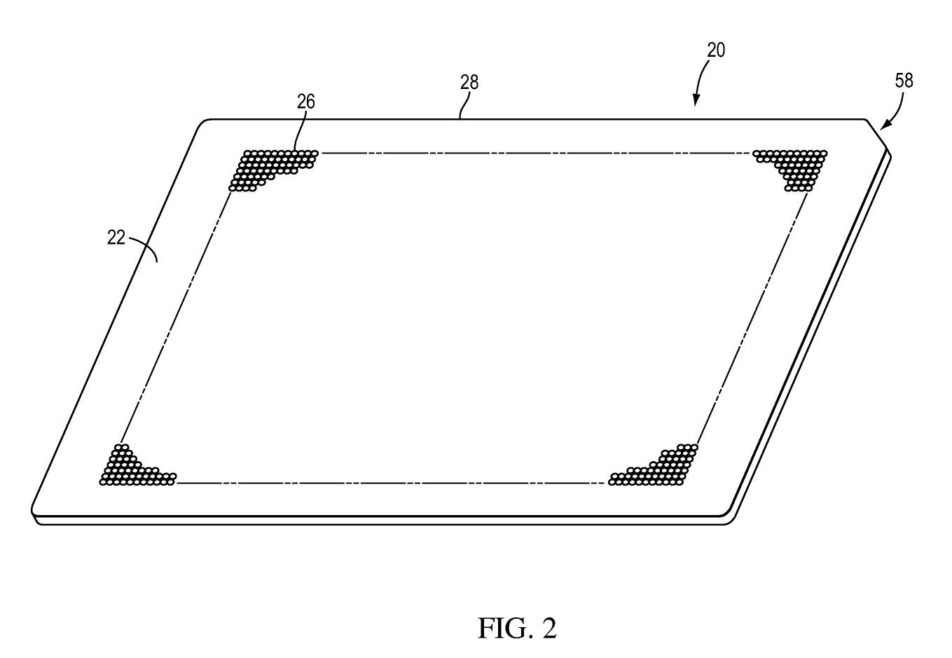

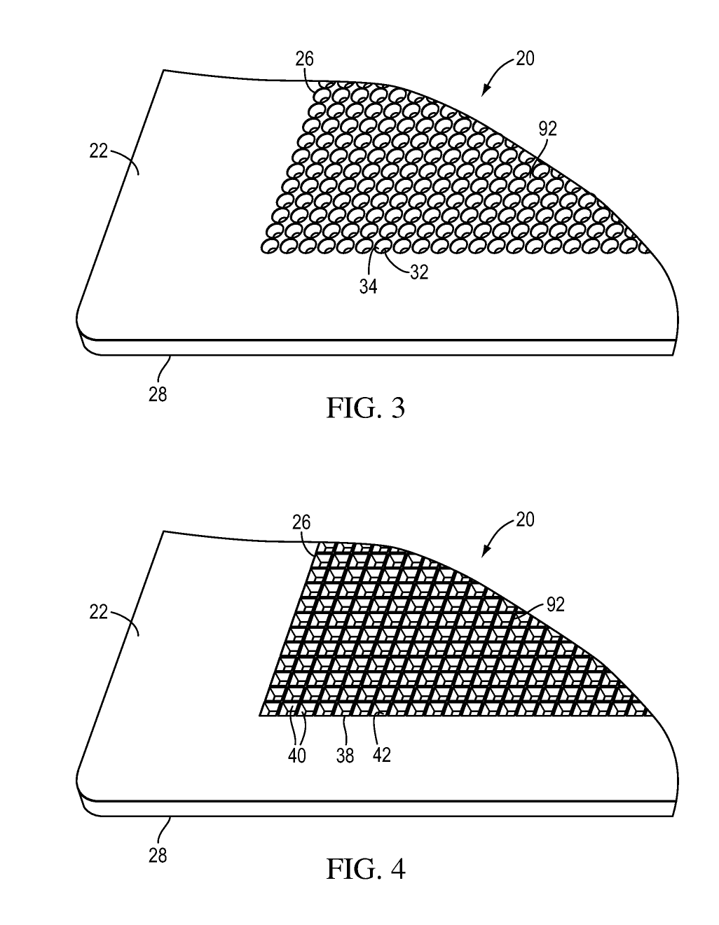

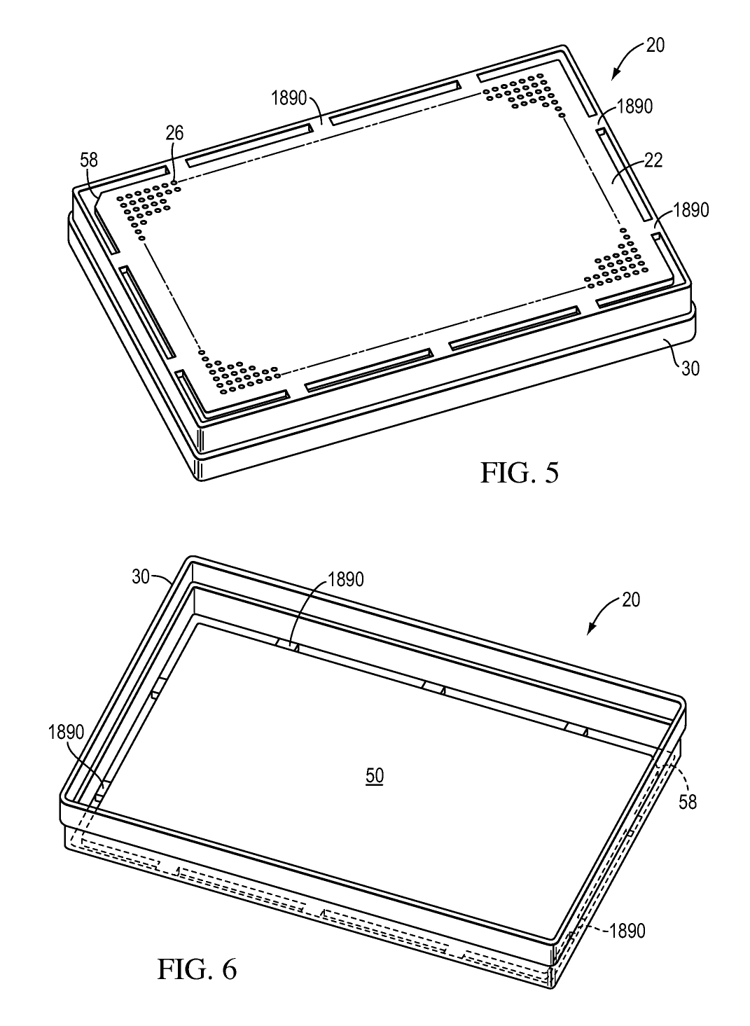

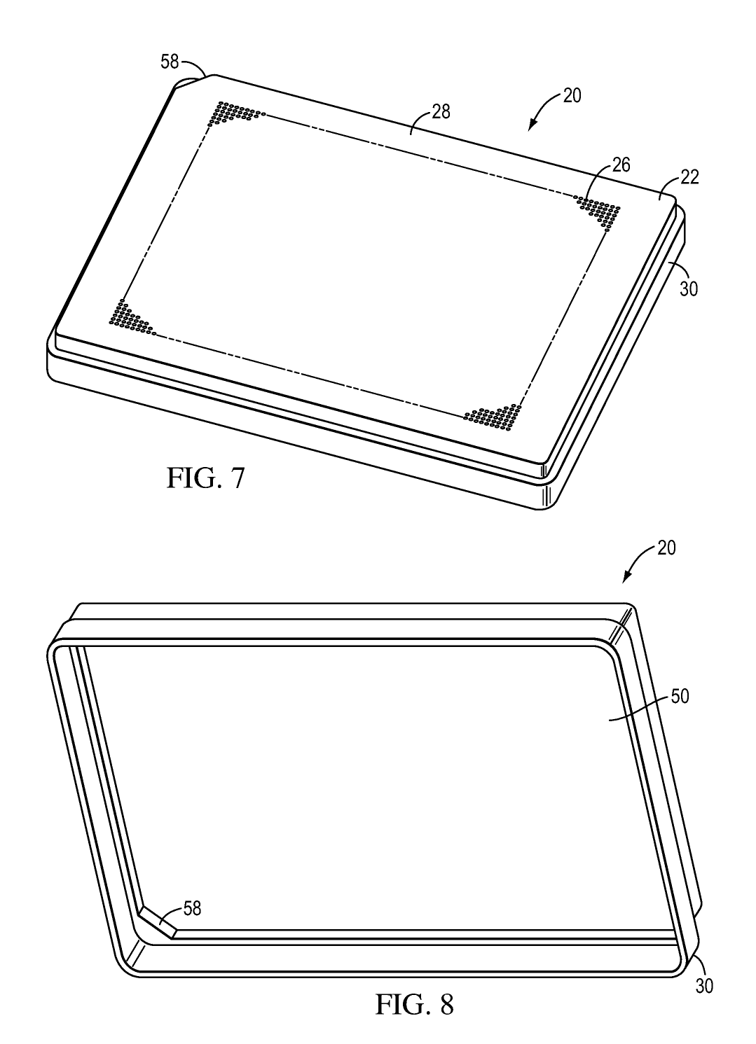

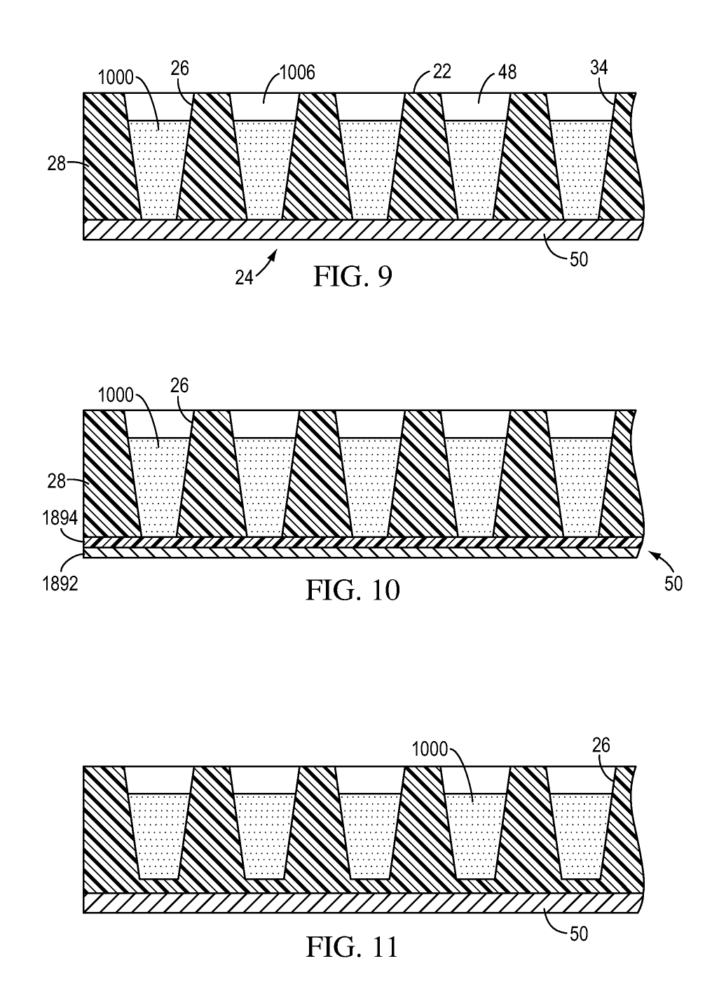

### DESCRIPTION OF SOME EMBODIMENTS

The following description of some embodiments is merely exemplary in nature and is in no way intended to limit the present teachings, applications, or uses. Although the present teachings will be discussed in some embodiments as relating to polynucleotide amplification, such as PCR, such discussion should not be regarded as limiting the present teaching to only such applications.

The section headings and sub-headings used herein are for general organizational purposes only and are not to be construed as limiting the subject matter described in any way.

### Analytical System Using Microplate

In some embodiments, a microplate apparatus is provided for use in PCR amplification, label detection, analytical methods and/or chemical reactions. Such processes may involve the use of biological materials including for example, tissues, cells/cell extracts, DNA, RNA, proteins, amino acids and other materials. Such microplates are, in some embodiments, useful in the analysis of assays, as further described below. The microplates may be configured for use in analyses/systems comprising an excitation system and a detection system and can be useful for analytical methods involving the generation and/or detection of electromagnetic radiation (e.g., visible, ultraviolet or infrared light) generated during analytical procedures. In some embodiments, the microplate may be used in assays including those comprising the use of fluorescent or other materials that absorb and/or emit light or other radiation under conditions that allow quantitative and/or qualitative analysis of a material (e.g., assays among those described herein). In some embodiments the microplate may be useful for polynucleotide amplification and/or detection and configured for use in connection with a thermocycler. In some embodiments, the microplate may be used with components for filling and handling the microplate. It will be understood that the microplates may be configured for use with a variety of assays, protocols and instrumentation, and such systems and components thereof are useful with a variety of analytical platforms, equipment, and procedures.

An analytical system may be used in connection with the microplate containing one or more assays; thermally regulated using a thermocycler system; illuminated or exposed to an excitation system; and assayed or sampled using a detection system. In some embodiments, the one or more assays can comprise any material that is useful in, the subject of, a precursor to, or a product of, an analytical method or chemical reaction. In some embodiments for amplification and/or detection of polynucleotides, assays comprise one or more reagents (such as PCR master mix, as described further herein); an analyte (such as a biological sample comprising DNA, a DNA fragment, cDNA, RNA, or any other nucleic acid sequence), one or more primers, one or more primer sets, one or more detection probes; components thereof; and combinations thereof. In some embodiments, assays comprise a homogenous solution of a DNA sample, at least one primer set, at least one detection probe, a polymerase, and a buffer, as used in a homogenous assay (described further herein). In some embodiments, assays can comprise an aqueous solution of at least one analyte, at least one primer set, at least one detection probe, and a polymerase. In some embodiments, assays can be an aqueous homogenous solution. In some embodiments, assays can comprise at least one of a plurality of different detection probes and/or primer sets to perform multiplex PCR, which can be useful, for example, when analyzing a whole genome (e.g., 20,000 to 30,000 genes, or more) or other large numbers of genes or sets of genes.

### Microplate Details

In some embodiments, a microplate comprises a substrate useful in the performance of an analytical method or chemical reaction. In some embodiments, the microplate is substantially planar, having substantially planar upper and lower surfaces, wherein the dimensions of the planar surfaces in the x- and y-dimensions are substantially greater than the thickness of the substrate in the z-direction. In some embodiments, a microplate can comprise one or more material retention regions or reaction chambers, configured to hold or support a material (e.g., an assay, as discussed below, or other solid or liquid) at one or more locations on or in the microplate. In some embodiments, such material retention regions can be wells, through-holes, reaction spots or pads, and the like. In some embodiments, such as shown in the Figures, material retention regions comprise wells, as at 26. In some embodiments, such wells can comprise a feature on or in the surface of the microplate wherein assays are contained at least in part by physical separation from adjacent features. Such well features can include, in some embodiments, depressions, indentations, ridges, and combinations thereof, in regular or irregular shapes. In some embodiments a microplate is single-use, wherein it is filled or otherwise used with a single assay for a single experiment or set of experiments, and is thereafter discarded. In some embodiments, a microplate is multiple-use, wherein it can be operable for use in a plurality of experiments or sets of experiments.

Referring now to the Figures, in some embodiments, microplate 20 comprises a substantially planar construction having a first surface 22 and an opposing second surface 24. First surface 22 comprises a plurality of wells 26 disposed therein or thereon. The overall positioning of the plurality of wells 26 can be referred to as a well array. Each of the plurality of wells 26 is sized to receive assays. As illustrated in the Figures, assays can be disposed in at least one of the plurality of wells 26 and optionally covered. In some embodiments, one or more of the plurality of wells 26 may not be completely filled with assay material, thereby defining a headspace which can define an air gap or other gas gap.

In some embodiments, the material retention regions of microplate 20 can comprise a plurality of reaction spots on the surface of the microplate. In such embodiments, a reaction spot can be an area on the microplate which localizes, at least in part by non-physical means, an assay. In such embodiments, an assay can be localized in sufficient quantity, and isolation from adjacent areas on the microplate, so as to facilitate an analytical or chemical reaction (e.g., amplification of one or more target DNA) in the material retention region. Such localization can be accomplished by physical and chemical modalities, including, for example, physical containment of reagents in one dimension and chemical containment in one or more other dimensions.

### Microplate Footprint

With reference to the Figures, microplate 20 generally comprises a main body or substrate 28. In some embodiments, main body 28 is substantially planar. In some embodiments, microplate 20 comprises an optional skirt or flange portion 30 disposed about a periphery of main body 28 (see Figures). Skirt portion 30 can form a lip around main body 28 and can vary in height. Skirt portion 30 can facilitate alignment of microplate 20 on thermocycler block 102. Additionally, skirt portion 30 can provide additional rigidity to microplate 20 such that during handling, filling, testing, and the like, microplate 20 remains rigid, thereby ensuring assay, or any other components, disposed in each of the plurality of wells 26 does not contaminate adjacent wells. However, in some embodiments, microplate 20 can employ a skirtless design (see Figures) depending upon user preference.

In some embodiments, microplate 20 can be from about 50 to about 200 mm in width, and from about 50 to about 200 mm in length. In some embodiments, microplate 20 can be from about 50 to about 100 mm in width, and from about 100 to about 150 mm in length. In some embodiments, microplate 20 can be about 72 mm wide and about 120 mm long.

In order to facilitate use with existing equipment, robotic implements, and instrumentation, the footprint dimensions of main body 28 and/or skirt portion 30 of microplate 20, in some embodiments, can conform to standards specified by the Society of Biomolecular Screening (SBS) and the American National Standards Institute (ANSI), published January 2004 (ANSI/SBS 3-2004). In some embodiments, the footprint dimensions of main body 28 and/or skirt portion 30 of microplate 20 are about 127.76 mm (5.0299 inches) in length and about 85.48 mm (3.3654 inches) in width. In some embodiments, the outside corners of microplate 20 comprise a corner radius of about 3.18 mm (0.1252 inches). In some embodiments, microplate 20 comprises a thickness of about 0.5 mm to about 3.0 mm. In some embodiments, microplate 20 comprises a thickness of about 1.25 mm. In some embodiments, microplate 20 comprises a thickness of about 2.25 mm. One skilled in the art will recognize that microplate 20 and skirt portion 30 can be formed in dimensions other than those specified herein.

### Plurality of Material Retention Regions

The density of material retention regions (i.e., number of material retention regions per unit surface area of microplate) and the size and volume of material retention regions can vary depending on the desired application and such factors as, for example, the species of the organism for which the methods of the present teachings may be employed. In some embodiments, the density of material retention regions can be varied as desired. In some embodiments, the pitch of material retention regions on microplate 20 can be varied as desired. In some embodiments, the distance between the material retention regions (the thickness of the wall between chambers) can be varied as desired. In some embodiments, the total number of material retention regions on the microplate can be varied as desired.

In order to increase throughput of genotyping, gene expression, and other assays, in some embodiments, microplate 20 comprises an increased quantity of the plurality of wells 26 beyond that employed in prior conventional microplates. In some embodiments, microplate 20 comprises 6,1536 wells. According to the present teachings, microplate 20 can comprise, but is not limited to, any of the array configurations of wells described in Table 1.

TABLE 1

| Total Number of Wells | Rows × Columns | Approximate Well Area | |
|---|---|---|---|
| 96 | 8 × 12 | 9 × 9 | mm |
| 384 | 16 × 24 | 4.5 × 4.5 | mm |
| 1536 | 32 × 48 | 2.25 × 2.25 | mm |
| 3456 | 48 × 72 | 1.5 × 1.5 | mm |
| 6144 | 64 × 96 | 1.125 × 1.125 | mm |
| 13824 | 96 × 144 | 0.75 × .075 | mm |
| 24576 | 128 × 192 | 0.5625 × 0.5625 | mm |
| 55296 | 192 × 288 | 0.375 × 0.375 | mm |
| 768 | 24 × 32 | 3 × 3 | mm |
| 1024 | 32 × 32 | 2.25 × 3 | mm |
| 1600 | 40 × 40 | 1.8 × 2.7 | mm |
| 1280 | 32 × 40 | 2.25 × 2.7 | mm |
| 1792 | 32 × 56 | 2.25 × 1.714 | mm |
| 2240 | 40 × 56 | 1.8 × 1.714 | mm |
| 864 | 24 × 36 | 3 × 3 | mm |
| 4704 | 56 × 84 | 1.257 × 1.257 | mm |
| 7776 | 72 × 108 | 1 × 1 | mm |
| 9600 | 80 × 120 | 0.9 × .09 | mm |
| 11616 | 88 × 132 | 0.818 × 0.818 | mm |
| 16224 | 104 × 156 | 0.692 × 0.692 | mm |
| 18816 | 112 × 168 | 0.643 × 0.643 | mm |
| 21600 | 120 × 180 | 0.6 × 0.6 | mm |
| 27744 | 136 × 204 | 0.529 × 0.529 | mm |
| 31104 | 144 × 216 | 0.5 × 0.5 | mm |
| 34656 | 152 × 228 | 0.474 × 0.474 | mm |
| 38400 | 160 × 240 | 0.45 × 0.45 | mm |
| 42336 | 168 × 252 | 0.429 × 0.429 | mm |
| 46464 | 176 × 264 | 0.409 × 0.409 | mm |
| 50784 | 184 × 256 | 0.391 × 0.391 | mm |

### Material Retention Region Size and Shape

According to some embodiments, as illustrated in the Figures, each of the plurality of material retention regions (e.g., wells 26) can be substantially equivalent in size. The plurality of wells 26 can have any cross-sectional shape. In some embodiments, as illustrated in the Figures, each of the plurality of wells 26 comprises a generally circular rim portion 32 with a downwardly-extending, generally-continuous sidewall 34 that terminate at a bottom wall 36 interconnected to sidewall 34 with a radius. A draft angle of sidewall 34 can be used in some embodiments. In some embodiments, the draft angle provides benefits including increased ease of manufacturing and minimizing shadowing (as discussed herein). The particular draft angle is determined, at least in part, by the manufacturing method and the size of each of the plurality of wells 26. In some embodiments, circular rim portion 32 can be about 1.0 mm in diameter, the depth of each of the plurality of wells 26 can be about 0.9 mm, the draft angle of sidewall 34 can be about 1° to 5° or greater and each of the plurality of wells 26 can have a center-to-center distance of about 1.125 mm. In some embodiments, the volume of each of the plurality of wells 26 can be about 500 nanoliters.

According to some embodiments, as illustrated in the Figures, each of the plurality of wells 26 comprises a generally square-shaped rim portion 38 with downwardly-extending sidewalls 40 that terminate at a bottom wall 42. A draft angle of sidewalls 40 can be used. Again, the particular draft angle is determined, at least in part, by the manufacturing method and the size of each of the plurality of wells 26. In some embodiments of wells 26, generally square-shaped rim portion 38 can have a side dimension of about 1.0 mm in length, a depth of about 0.9 mm, a draft angle of about 1° to 5° or greater, and a center-to-center distance of about 1.125 mm, generally indicated at A (see Figures). In some embodiments, the volume of each of the plurality of wells 26 can be about 500 nanoliters. In some embodiments, the spacing between adjacent wells 26, as measured at the top of a wall dividing the wells, is less than about 0.5 m. In some embodiments, this spacing between adjacent wells 26 is about 0.25 mm.

In some embodiments, and in some configurations, the plurality of wells 26 comprising a generally circular rim portion 32 can provide advantages over the plurality of wells 26 comprising a generally square-shaped rim portion 38. In some embodiments, during heating, it has been found that assays can migrate through capillary action upward along edges of sidewalls 40. This can draw the assay from the center of each of the plurality of wells 26, thereby causing variation in the depth of the assays. Variations in the depth of an assay can influence the emission output of assays during analysis. Additionally, during manufacture of microplate 20, in some cases cylindrically shaped mold pins used to form the plurality of wells 26 comprising generally circular rim portion 32 can permit unencumbered flow of molten polymer thereabout. This unencumbered flow of molten polymer results in less deleterious polymer molecule orientation. In some embodiments, generally circular rim portion 32 provides more surface area along microplate 20 for improved sealing with sealing cover 80, as is discussed herein.

In some embodiments, the area of each material retention region can be varied as desired. In some embodiments, the width of each material retention region can be from about 200 to about 2,000 microns, or from about 800 to about 3000 microns. In some embodiments, the depth of each material retention region can be varied as desired. In some embodiments, the surface area of each material retention region can be varied as desired. In some embodiments, the aspect ratio (ratio of depth:width) of each material retention region can be varied as desired.

In some embodiments, the volume of the material retention regions can be less than about 50 μl, or less than about 10 μl. In some embodiments, the volume can be from about 0.05 to about 500 nanoliters, from about 0.1 to about 200 nanoliters, from about 20 to about 150 nanoliters, from about 80 to about 120 nanoliters, from about 50 to about 100 nanoliters, from about 1 to about 5 nanoliters, or less than about 2 nanoliters.

### Through-Hole Material Retention Regions

As illustrated in the Figures, in some embodiments, each of the material retention regions of microplate 20 can comprise a plurality of apertures 48 being sealed at least on one end by sealing cover 80 or other sealing member, such as a thermally conductive member (discussed in greater detail herein). In some embodiments, each of the plurality of apertures 48 can be sealed on an opposing end with a backing sheet 50, which can have a clear or opaque adhesive or can be coupled in alternative process to be discussed herein. In some embodiments, as illustrated in FIG. 68-70, backing sheet 50 can comprise a heat conducting material such as, for example, a metal foil or a metal coated plastic. In some embodiments, backing sheet 50 can be placed against thermocycler block 102 to aid in thermal conductivity and distribution. In some embodiments, backing sheet 50 can comprise a plurality of reaction spots (as discussed herein), coated on discrete areas of the surface of backing sheet 50, such that in some circumstances the plurality of reaction spots can be aligned with the plurality of apertures 48.

In some embodiments, a layer of mineral oil can be placed at the top of each of the plurality of apertures 48 before, or as an alternative to, placement of sealing cover 80 on microplate 20. In several of such embodiments, the mineral oil can fill a portion of each of the plurality of apertures 48 and provide an optical interface and can control evaporation of the assay.

### Grooves

In some embodiments, microplate 20 can comprise grooves disposed about a periphery of the plurality of wells 26. In some embodiments, grooves can have depth and width dimensions generally similar to the depth and width dimensions of the plurality of wells 26. In some embodiments, grooves can have depth and width dimensions less than the depth and width dimensions of the plurality of wells 26. In some embodiments, additional grooves can be disposed at opposing sides of microplate 20. In some embodiments, grooves can improve thermal uniformity among the plurality of wells 26 in microplate 20. In some embodiments, grooves can improve the sealing interface formed by sealing cover 80 and microplate 20. Grooves can also assist in simplifying the injection molding process of microplate 20. In some embodiments, a liquid solution similar to an assay can be disposed in grooves to, in part, improve thermal uniformity during thermocycling.

### Alignment Features

In some embodiments, as illustrated in the Figures, microplate 20 comprises an alignment feature 58, such as a corner chamfer, a pin, a slot, a cut corner, an indentation, a graphic, or other unique feature that is capable of interfacing with a corresponding feature formed in a fixture, reagent dispensing equipment, and/or thermocycler. In some embodiments, alignment feature 58 comprises a nub or protrusion 60. Additionally, in some embodiments, alignment features 58 are placed such that they do not interfere with sealing cover or at least one of the plurality of wells 26. However, locating alignment features 58 near at least one of the plurality of wells 26 can provide improved alignment with dispensing equipment and/or thermocycler block.

### Thermally Isolated Sections

In some embodiments, as illustrated in the Figures, microplate 20 comprises a thermally isolated portion 62. Thermally isolated portion 62 can be disposed along at least one edge of main body 28. Thermally isolated portion 62 can be generally free of wells 26 and can be sized to receive a marking indicia 64 (discussed in detail herein) thereon. Thermally isolated portion 62 can further be sized to facilitate the handling of microplate 20 by providing an area that can be easily gripped by a user or mechanical device without disrupting the plurality of wells 26.

In some embodiments, microplate 20 comprises a first groove 66 formed along first surface 22 and a second groove 68 formed along an opposing second surface 24 of microplate 20. First groove 66 and second groove 68 can be aligned with respect to each other to extend generally across microplate 20 from a first side 70 to a second side 72. First groove 66 and second groove 68 can be further aligned upon first surface 22 and second surface 24 to define a reduced cross-section 74 between thermally isolated portion 62 and the plurality of wells 26. This reduced cross-section 74 can provide a thermal isolation barrier to reduce any heat sink effect introduced by thermally isolated portion 62, which might otherwise reduce the temperature cycle of some of the plurality of wells 26.

In some embodiments, as illustrated in the Figures, microplate 20 comprises main body 28 generally thermally isolated from skirt portion 30 via a series of alternating gap portions 1888 and support webs 1890. Gap portions 1888 lack material or structure. That is, in some embodiments, main body 28 supports the plurality of wells 26 and is generally thermally isolated from skirt portion 30 by virtue of the limited physical connections extending therebetween. By limiting the physical connection between main body 28 and skirt portion 30, heat transfer between main body 28 and skirt portion 30 is limited. Therefore, heating and/or cooling of the plurality of wells 26 and consequently assays contained therein can be more quickly and accurately controlled during thermocycling. As illustrated in the Figures, in some embodiments, microplate 20 can comprise a pair of support webs 1890 along a width of main body 28 and three support webs 1890 along a length of main body 28. In some embodiments, support webs 1890 can be thin and flexible to minimize thermal transfer between main body 28 and skirt portion 30 and further permit flexing of main body 28 during clamping from pressure clamp system 110 without being effected by the stiffer skirt portion 30. In some embodiments, support webs 1890 are formed from a different material than that of main body 28 and/or skirt portion 30 to provide desired mechanical or thermal characteristics.

### Marking Indicia

In some embodiments, as illustrated in the Figures, microplate 20 comprises marking indicia 64, such as graphics, printing, lithograph, pictorial representations, symbols, bar codes, handwritings or any other type of writing, drawings, etchings, indentations, embossments or raised marks, machine readable codes (i.e. bar codes, etc.), text, logos, colors, and the like. In some embodiments, marking indicia 64 is permanent.

In some embodiments, marking indicia 64 can be printed upon microplate 20 using any known printing system, such as inkjet printing, pad printing, hot stamping, and the like. In some embodiments, such as those using a light-colored microplate 20, a dark ink can be used to create marking indicia 64 or vice versa.

In some embodiments, microplate 20 can be made of polypropylene and have a surface treatment applied thereto to facilitate applying marking indicia 64. In some embodiments, such surface treatment comprises flame treatment, corona treatment, treating with a surface primer, or acid washing. However, in some embodiments, a UV-curable ink can be used for printing on polypropylene microplates.

Still further, in some embodiments, marking indicia 64 can be printed upon microplate 20 using a CO₂ laser marking system. Laser marking systems evaporate material from a surface of microplate 20. Because CO₂ laser etching can produce reduced color changes of marking indicia 64 relative to the remaining portions of microplate 20, in some embodiments, a YAG laser system can be used to provide improved contrast and reduced material deformation.

In some embodiments, a laser activated pigment can be added to the material used to form microplate 20 to obtain improved contrast between marking indicia 64 and main body 28. In some embodiments, an antimony-doped tin oxide pigment can be used, which is easily dispersed in polymers and has marking speeds as high as 190 inches per second. Antimony-doped tin oxide pigments can absorb laser light and can convert laser energy to thermal energy in embodiments where indicia are created using a YAG laser.

In some embodiments, marking indicia 64 can identify microplates 20 to facilitate identification during processing. Furthermore, in some embodiments, marking indicia 64 can facilitate data collection so that microplates 20 can be positively identified to properly correlate acquired data with the corresponding assay. Such marking indicia 64 can be employed as part of Good Laboratory Practices (GLP) and Good Manufacturing Practices (GMP), and can further, in some circumstances, reduce labor associated with manually applying adhesive labels, manually tracking microplates, and correlating data associated with a particular microplate.

In some embodiments, marking indicia 64 can assist in alignment by placing a symbol or other machine-readable graphic on microplate 20. An optical sensor or optical eye can detect marking indicia 64 and can determine a location of microplate 20. In some embodiments, such location of microplate 20 can then be adjusted to achieve a predetermined position using, for example, a drive system of sequence detection system 10, sealing cover applicator 1100, or other corresponding systems.

In some embodiments, the type (physical properties, characteristics, etc.) of marking indicia employed on a microplate can be selected so as to reduce thermal and/or chemical interference during thermocycling relative to what might otherwise occur with other types of marking indicia (e.g., common prior indicia designs, such as adhesive labels). For example, adhesive labels can, in some circumstances, interfere (e.g., chemically interact) with one or more reagents (e.g., dyes) being used.

Referring to the Figures, in some embodiments, a radio frequency identification (RFID) tag 76 can be used to electronically identify microplate 20. RFID tag 76 can be attached or molded within microplate 20. An RFID reader (not illustrated) can be integrated into sequence detection system 10 to automatically read a unique identification and/or data handling parameters of microplate 20. Further, RFID tag 76 does not require line-of-sight for readability. It should be appreciated that RFID tag 76 can be variously configured and used according to various techniques, such as those described in commonly-assigned U.S. patent application entitled “SAMPLE CARRIER DEVICE INCORPORATING RADIO FREQUENCY IDENTIFICATION, AND METHOD” (Attorney Docket No. 5010-193).

In some embodiments, as illustrated in the Figures, microplate 20 can comprise a multi-piece construction having main body 28 and backing 50. In some embodiments, backing 50 is a thermally conductive member. In some embodiments as illustrated in FIG. 69, backing 50 comprises a laminate having a metallic foil layer 1892 and a backing substrate 1894 coupled thereto. Such bonding of foil layer 1892 and polypropylene film 1894 can be completed through any known bonding method, such as pressure sensitive adhesive, thermoset adhesive, and other known methods. In some embodiments, metallic foil layer 1892 is a 4 mil aluminum foil and backing substrate 1894 is a 4 mil black polypropylene material. In some embodiments, metallic foil layer 1892 is position below backing substrate 1894 so as to be in contact with thermocycler system 100. However, in some embodiments, metallic foil layer 1892 is positioned above backing substrate 1894. Backing substrate 1894 can be larger than metallic foil layer 1892 to completely encapsulate metallic foil layer 1892 following coupled with main body 28 for additional protection of metallic foil layer 1892.

### Microplate Material

In some embodiments, microplate 20 can comprise, at least in part, a thermally conductive material. In some embodiments, a microplate, in accordance with the present teachings, can be molded, at least in part, of a thermally conductive material to define a cross-plane thermal conductivity of at least about 0.30 W/mK or, in some embodiments, at least about 0.58 W/mK. Such thermally conductive materials can provide a variety of benefits, such as, in some cases, improved heat distribution throughout microplate 20, so as to afford reliable and consistent heating and/or cooling of the assay. In some embodiments, this thermally conductive material comprises a plastic formulated for increased thermal conductivity. Such thermally conductive materials can comprise, for example and without limitation, at least one of polypropylene, polystyrene, polyethylene, polyethyleneterephthalate, styrene, acrylonitrile, cyclic polyolefin, syndiotactic polystyrene, polycarbonate, liquid crystal polymer, conductive fillers or plastic materials; and mixtures or combinations thereof. In some embodiments, such thermally conductive materials include those known to those skilled in the art with a melting point greater than about 130° C. For example, microplate 20 can be made of commercially available materials such as RTP199X104849, COOLPOLY E1201, or, in some embodiments, a mixture of about 80% RTP199X104849 and 20% polypropylene.

In some embodiments, microplate 20 can comprise at least one carbon filler, such as carbon, graphite, impervious graphite, and mixtures or combinations thereof. In some cases, graphite has an advantage of being readily and cheaply available in a variety of shapes and sizes. One skilled in the art will recognize that impervious graphite can be non-porous and solvent-resistant. Progressively refined grades of graphite or impervious graphite can provide, in some cases, a more consistent thermal conductivity.

In some embodiments, one or more thermally conductive ceramic fillers can be used, at least in part, to form microplate 20. In some embodiments, the thermally conductive ceramic fillers can comprise boron nitrate, boron nitride, boron carbide, silicon nitride, aluminum nitride, and mixtures or combinations thereof.

In some embodiments, microplate 20 can comprise an inert thermally conductive coating. In some embodiments, such coatings can include metals or metal oxides, such as copper, nickel, steel, silver, platinum, gold, copper, iron, titanium, alumina, magnesium oxide, zinc oxide, titanium oxide, and mixtures thereof.

In some embodiments, microplate 20 comprises a mixture of a thermally conductive material and other materials, such as non-thermally conductive materials or insulators. In some embodiments, the non-thermally conductive material comprises glass, ceramic, silicon, standard plastic, or a plastic compound, such as a resin or polymer, and mixtures thereof to define a cross-plane thermal conductivity of below about 0.30 W/mK. In some embodiments, the thermally conductive material can be mixed with liquid crystal polymers (LCP), such as wholly aromatic polyesters, aromatic-aliphatic polyesters, wholly aromatic poly(ester-amides), aromatic-aliphatic poly(ester-amides), aromatic polyazomethines, aromatic polyester-carbonates, and mixtures thereof. In some embodiments, the composition of microplate 20 can comprise from about 30% to about 60%, or from about 38% to about 48% by weight, of the thermally conductive material.

The thermally conductive material and/or non-thermally conductive material can be in the form of, for example, powder particles, granular powder, whiskers, flakes, fibers, nanotubes, plates, foil layers, sheets, rice, strands, hexagonal or spherical-like shapes, or any combination thereof. In some embodiments, the microplate comprises thermally conductive additives having different shapes to contribute to an overall thermal conductivity that is higher than any one of the individual additives alone.

In some embodiments, the thermally conductive material comprises a powder. In some embodiments, the particle size used herein can be between 0.10 micron and 300 microns. When mixed homogeneously with a resin in some embodiments, powders provide uniform (i.e. isotropic) thermal conductivity in all directions throughout the composition of the microplate.

As discussed above, in some embodiments, the thermally conductive material can be in the form of flakes. In some such embodiments, the flakes can be irregularly shaped particles produced by, for example, rough grinding to a desired mesh size or the size of mesh through which the flakes can pass. In some embodiments, the flake size can be between 1 micron and 200 microns. Homogenous compositions containing flakes can, in some cases, provide uniform thermal conductivity in all directions.

In some embodiments, the thermally conductive material can be in the form of fibers, also known as rods. Fibers can be described, among other ways, by their lengths and diameters. In some embodiments, the length of the fibers can be, for example, between 2 mm and 15 mm. The diameter of the fibers can be, for example, between 1 mm and 5 mm. Formulations that include fibers in the composition can, in some cases, have the benefit of reinforcing the resin for improved material strength.

In some embodiments, microplate 20 can comprise a material comprising additives to promote other desirable properties. In some embodiments, these additives can comprise flame-retardants, antioxidants, plasticizers, dispersing aids, marking additives, and mold-releasing agents. In some embodiments, such additives are biologically and/or chemically inert.

In some embodiments, microplate 20 comprises, at least in part, an electrically conductive material, which can improve reagent dispensing alignment. In this regard, electrically conductive material can reduce static build-up on microplate 20 so that the reagent droplets will not go astray during dispensing. In some embodiments, a voltage can be applied to microplate 20 to pull the reagent droplets into a predetermined position, particularly with a co-molded part where the bottom section can be electrically conductive and the sides of the plurality of wells 26 may not be electrically conductive. In some embodiments, a voltage field applied to the electrically conductive material under the well or wells of interest can pull each assay into the appropriate wells.

In some embodiments, microplate 20 can be made, at least in part, of non-electrically conductive materials. In some embodiments, non-electrically conductive materials can at least in part comprise one or more of crystalline silica (3.0 W/mK), aluminum oxide (42 W/mK), diamond (2000 W/mK), aluminum nitride (150-220 W/mK), crystalline boron nitride (1300 W/mK), and silicon carbide (85 W/mK).

### Microplate Molding

In some embodiments, microplate 20 can be molded by first extruding a melt blend comprising a mixture of a polymer and one or more thermally conductive materials and/or additives. In some embodiments, the polymer and thermally conductive additives can be fed into a twin-screw extruder using a gravimetric feeder to create a well-dispersed melt blend. In some embodiments, the extruded melt blend can be transferred through a water bath to cool the melt blend before being pelletized and dried. The pelletized melt blend can then be heated above its melting point by an injection molding machine and then injected into a mold cavity. The mold cavity can generally conform to a desired shape of microplate 20. In some embodiments, the injection-molding machine can cool the injected melt blend to create microplate 20. Finally, microplate 20 can be removed from the injection-molding machine.

In some embodiments, two or more material types of pellets can be mixed together and the combination then placed in the injection molding machine to be melt blended during the injection molding process. In some embodiments, microplate 20 can be molded by first receiving pellet material from a resin supplier; drying the pellet material in a resin dryer; transferring the dried pellet material with a vacuum system into a hopper of a mold press; molding microplate 20; trimming any resultant gates or flash; and packaging microplate 20. In some embodiments, the mold cavity can be centrally gated along the second surface 24 of microplate 20. In some embodiments, the mold cavity can be gated along a perimeter of main body 28 and/or skirt portion 30 of microplate 20.

Referring to the Figures, in some embodiments, main body 28 of microplate 20 is formed to include the plurality of through holes 48 extending therethrough—that is, the plurality of through holes 48 are enclosed at a bottom thereof. In such arrangements, backing 50 can be coupled to main body 28 to, at least in part, seal or otherwise enclose the plurality of through holes 48 to form the plurality of wells 26. However, main body 28 can comprise a self-contained, self-defined plurality of wells 26 in that a bottom structure is formed to prevent a through-hole configuration.

Backing 50 can be coupled to main body 28, in some embodiments, through the use of insert molding. In insert molding, backing 50, which can comprise either a single material or a laminate as described herein, can be placed within a mold cavity prior to injection of molten material. Upon injection of molten molding material into the mold cavity, melting and later bonding of the injected material with the material of backing 50 can be completed.

In some embodiments, backing 50 can be coupled to main body 28 through laser welding. In such arrangement, a laser source can be used to emit a laser beam. Depending upon the particular configuration of backing 50, the laser source can be positioned either above or below main body 28 and backing 50 and the materials thereof can be selected to permit the laser beam to enter one of the main body 28 and backing 50 and pass to a weld zone. For example, if the laser source is positioned above main body 28 and backing 50, the material of main body 28 can be selected to be transmissive to the laser beam while the material of backing 50 can be absorptive to the laser beam. As the laser beam passes through main body 28 it impacts backing 50. As a result of backing 50 being made of an absorptive material, backing 50 is heated to a melting point of main body 28 and/or backing 50 along a weld zone between main body 28 and backing 50. The resultant molten material near the weld zone then bonds or otherwise fuses to cause main body 28 and backing 50 to be welded together once cooled below the melting point. In some embodiments, backing 50 can be coupled to main body 28 through ultrasonic welding, film decorating-type processing within the injection mold, or similar processes.

### Methods of Use and Analysis

### Polynucleotide Amplification

In some embodiments, the microplate may be used in a system for the amplification of polynucleic acids, such as by PCR. Briefly, by way of background, PCR can be used to amplify a sample of target analyte, such as, for example, target Deoxyribose Nucleic Acid (DNA), for analysis. Typically, the PCR reaction involves copying the strands of the target DNA and then using the copies to generate additional copies in subsequent cycles. Each cycle doubles the amount of the target DNA present, thereby resulting in a geometric progression in the number of copies of the target DNA. The temperature of a double-stranded target DNA is elevated to denature the DNA, and the temperature is then reduced to anneal at least one primer to each strand of the denatured target DNA. In some embodiments, the target DNA can be a cDNA. In some embodiments, primers are used as a pair—a forward primer and a reverse primer—and can be referred to as a primer pair or primer set. In some embodiments, the primer set comprises a 5′ upstream primer that can bind with the 5′ end of one strand of the denatured target DNA and a 3′ downstream primer that can bind with the 3′ end of the other strand of the denatured target DNA. Once a given primer binds to the strand of the denatured target DNA, the primer can be extended by the action of a polymerase. In some embodiments, the polymerase can be a thermostable DNA polymerase, for example, a Taq polymerase. The product of this extension, which sometimes may be referred to as an amplicon, can then be denatured from the resultant strands and the process can be repeated. Temperatures suitable for carrying out the reactions are well known in the art. Certain basic principles of PCR are set forth in U.S. Pat. Nos. 4,683,195, 4,683,202, 4,800,159, and 4,965,188, each issued to Mullis et al.

In some embodiments, PCR can be conducted under conditions allowing for quantitative and/or qualitative analysis of one or more target DNA. Accordingly, detection probes can be used for detecting the presence of the target DNA in an assay. In some embodiments, the detection probes can comprise physical (e.g., fluorescent) or chemical properties that change upon binding of the detection probe to the target DNA. Some embodiments of the present teaching can provide real time fluorescence-based detection and analysis of amplicons as described, for example, in PCT Publication No. WO 95/30139 and U.S. patent application Ser. No. 08/235,411.

In some embodiments, assays can be a plurality of homogenous polynucleotide amplification assays, for coupled amplification and detection, wherein the process of amplification generates a detectable signal and the need for subsequent sample handling and manipulation to detect the amplified product is minimized or eliminated. Homogeneous assays can provide for amplification that is detectable without opening a sealed well or further processing steps once amplification is initiated. Such homogeneous assays can be suitable for use in conjunction with detection probes. For example, in some embodiments, the use of an oligonucleotide detection probe, specific for detecting a particular target DNA can be included in an amplification reaction in addition to a DNA binding agent of the present teachings. Homogenous assays among those useful herein are described, for example, in commonly assigned U.S. Pat. No. 6,814,934.

In some embodiments, methods are provided for detecting a plurality of targets. Such methods include those comprising forming an initial mixture comprising an analyte sample suspected of comprising the plurality of targets, a polymerase, and a plurality of primer sets. In some embodiments, each primer set comprises a forward primer and a reverse primer and at least one detection probe unique for one of the plurality of primer sets. In some embodiments, the initial mixture can be formed under conditions in which one primer elongates if hybridized to a target.

In some embodiments, the location of a fluorescent signal on a solid support, such as microplate 20, can be indicative of the identity of a target comprised by the analyte sample. In some embodiments, a plurality of detection probes are distributed to identify loci of at least some of the plurality of wells 26 of microplate 20. A signal deriving from a detection probe, such as, for example, an increase in fluorescence intensity of a fluorophore at a particular locus can be detected if an amplification product binds to a detection probe and is then amplified. The location of the locus can indicate the identity of the target, and the intensity of the fluorescence can indicate the quantity of the target.

In some embodiments, reagents are provided comprising a master mix comprising at least one of catalysts, initiators, promoters, cofactors, enzymes, salts, buffering agents, chelating agents, and combinations thereof. In some embodiments, reagents can include water, a magnesium catalyst (such as MgCl₂), polymerase, a buffer, and/or dNTP. In some embodiments, specific master mixes can comprise AmpliTaq® Gold PCR Master Mix, TaqMan® Universal Master Mix, TaqMan® Universal Master Mix No AmpErase® UNG, Assays-by-DesignSM, Pre-Developed Assay Reagents (PDAR) for gene expression, PDAR for allelic discrimination and Assays-On-Demand®, (all of which are marketed by Applied Biosystems). However, the present teachings should not be regarded as being limited to the particular chemistries and/or detection methodologies recited herein, but may employ Taqman®; Invader®; Taqman Gold®; protein, peptide, and immuno assays; receptor binding; enzyme detection; and other screening and analytical methodologies.

In some embodiments, sequence detection system 10 is operable for analysis of assays (e.g., polynucleotides) comprising or derived from genetic materials from organisms. In some embodiments, assays comprise substantially all of the genetic material from an organism, such as, for example, a human, mouse, rat, dog, rabbit, primate or any other mammal, bacteria, Arabidopsis or any other plants, insect, fungus, yeast and virus, including sub-species, strains, and individual subject organisms thereof. In some embodiments, assays comprise at least one of a homogenous solution of a DNA sample, at least one primer set for detection of a polynucleotide comprising or derived from such genetic materials, at least one detection probe, a polymerase, and a buffer. In some embodiments, assays comprise at least one of a plurality of different detection probes and/or primer sets to perform multiplex PCR, which can be particularly useful when analyzing a whole genome having, for example, about 30,000 different genes. In some embodiments, analysis of substantially the entire genome of an organism is conducted on a single microplate 20, or on multiple microplates (e.g., two, three, four or more) each comprising subparts of such materials comprising or derived from the genetic materials of the organism. In some embodiments using multiple microplates, a plurality of plates contain a plurality of assays having essentially identical materials and a plurality of assays having different materials. In some embodiments, a plurality of plates do not contain assays having essentially identical materials. In some embodiments, microplate 20 comprises a fixed subset of a genome. It should also be recognized that the present teachings can be used in connection with genotyping, gene expression, or other analysis.

The present teachings can further be used in connection with the teachings of commonly-assigned U.S. patent application Ser. No. 11/087,101.

What is claimed is:

1. A microplate comprising:
a main body portion having a first surface and an opposing second surface;
a plurality of through holes formed in said main body portion, each through hole being disposed about a respective axis normal to said second surface; and
a backing coupled to the main body portion, said backing including a first layer comprising an adhesive, a second layer comprising a backing material coupled to said main body portion via said adhesive, and a third layer comprising a thermally conductive material coupled to said second layer, said second layer and third layer each covering said plurality of through holes so that each of said respective axes passes through said backing material and through said thermally conductive material.

2. The microplate according to claim 1, wherein the backing is coupled to said opposing second surface of said main body portion.

3. The microplate according to claim 1 wherein said backing is a laminate.

4. The microplate according to claim 1 wherein said thermally conductive material is a metallic foil.

5. The microplate according to claim 1 wherein said backing further comprises an adhesive coupling together said second layer and said third layer.

6. The microplate according to claim 1 wherein said backing further comprises a weld that couples said backing to said main body portion.

7. The microplate according to claim 6 wherein said weld is an ultrasonic weld or a laser weld.

8. The microplate according to claim 1 wherein said backing is made of one of a material transmissive to laser energy or a material absorptive to laser energy, said main body portion being made of the other of said material transmissive to laser energy or said material absorptive to laser energy, said backing being coupled to said main body portion via laser welding.

9. The microplate according to claim 1 wherein said plurality of through holes is at least 1536 through holes.

10. The microplate according to claim 1 further comprising a skirt portion.

11. The microplate according to claim 1 further comprising a layer of oil disposed at a top of each said plurality of through holes.

12. The microplate according to claim 11 wherein said oil provides an optical interface.

13. The microplate according to claim 12 further comprising an assay disposed inside each of said plurality of through holes, wherein said oil covers said assay.

14. The microplate according to claim 13 wherein said oil controls evaporation of said assay.

15. The microplate according to claim 1 wherein said plurality of through holes and said backing together form a plurality of wells, each well including one or more sides, said backing being made of an electrically conductive material and said sides being electrically non-conductive.

16. The microplate according to claim 15 wherein said microplate is configured to pull reagent into said plurality of wells when a voltage is applied to said microplate.

17. The microplate according to claim 1 wherein each through hole is centered about the respective axis normal thereof.

18. A microplate comprising:
a main body portion extending along a plane;
a plurality of through holes formed in said main body portion, each through hole being disposed about a respective axis normal to said plane; and
a backing coupled to said main body portion, said backing including a first layer comprising a backing material coupled to said main body portion, a second layer comprising a thermally conductive material coupled to said second layer, and an adhesive layer coupling together said first layer and said second layer, said first layer and said second layer each masking said plurality of through holes so that each of said respective axes passes through said backing material and through said thermally conductive material.

19. The microplate according to claim 18 wherein said backing further comprises a weld that couples said backing to said main body portion.

20. The microplate according to claim 19 wherein said weld is an ultrasonic weld or a laser weld.
